(19) United States
(12) Patent Application Publication
(10) Pub. No.: US 2026/0262989 A1
(43) Pub. Date: Sep. 10, 2026

(54) MULTIELECTRODE ARRAY FOR INTRAOPERATIVE ENDOCARDIAL CONDUCTION MAPPING

(71) Applicant: The Children's Medical Center Corporation, Boston, MA (US)

(72) Inventors: Eric N. Feins, Boston, MA (US); Jocelyn Davee, South Boston, MA (US); Elizabeth Dewitt, Boston, MA (US); Edward O'Leary, Boston, MA (US); Sitaram Emani, Boston, MA (US)

(73) Assignee: The Children's Medical Center Corporation, Boston, MA (US)

(21) Appl. No.: 19/163,482

(22) PCT Filed: Jul. 14, 2023
(86) PCT No.: PCT/US2023/027800

§ 371 Date: Sep. 9, 2025

Related U.S. Application Data
(60) Provisional application No. 63/451,350, filed on Mar. 10, 2023.

(51) Int. Cl. A61B 5/287; A61B 5/367

(52) U.S. Cl. CPC A61B 5/287; A61B 5/367; A61B 2503/06; A61B 2562/046; A61B 2562/164; A61B 2562/227

(57) ABSTRACT

A system to map a cardiac conduction system during open heart surgery includes an electrode support and electrodes held in a fixed arrangement by the electrode support. The system also includes a flexible intermediate component. The electrodes and the electrode support are affixed to a first end of the intermediate component. A rigid handle is attached to a second end of the intermediate component, opposite the first end.

### RELATED APPLICATIONS

This application claims the benefit of priority under 35 U.S.C. § 119(e) to U.S. Provisional Application Ser. No. 63/451,350 filed Mar. 10, 2023 under Attorney Docket No. C1233.70270US00, which is hereby incorporated herein by reference in its entirety.

### BACKGROUND

### Field

Aspects of the present application relate to a multielectrode array for intraoperative endocardial conduction mapping and methods of using the same.

### Related Art

Open heart surgery involves accessing the heart through an opening in the chest and may be performed to address problems including plaque buildup, faulty heart valves, and abnormal heart rhythms. In children, open heart surgery may be needed to address congenital heart disease. While open heart surgery may be necessary and beneficial with regard to the problem being addressed by the procedure, the surgery itself may create additional complications. For example, mechanical injury to specialized conduction tissue inside the heart during a procedure to address congenital heart disease in a child may cause iatrogenic heart block, which may result in the child requiring a permanent pacemaker and lifelong ventricular pacing. The need for a permanent pacemaker may commit the child to numerous reoperations and interventional procedures. In addition, the pacemaker may lead to complications such as infection, cardiac strangulation, and coronary artery compression from overlying pacemaker leads, an issue that can lead to sudden death. Chronic ventricular pacing can also cause deterioration in ventricular function over time. Generally, the economic and personal burden imposed by heart block can be enormous.

### SUMMARY

According to one or more embodiments, a system to map a cardiac conduction system during open heart surgery includes an electrode support and electrodes held in a fixed arrangement by the electrode support. The system also includes a flexible intermediate component. The electrodes and the electrode support are affixed to a first end of the intermediate component. A rigid handle is attached to a second end of the intermediate component, opposite the first end.

Additionally in one or more embodiments, a handheld cardiac conduction mapping system includes a rigid handle and a flexible intermediate component coupled to the handle. A set of probing portions include at least one probing portion. Each probing portion of the set of probing portions includes an array of electrodes, and an electrode support. The array of electrodes is encapsulated in the electrode support.

According to another embodiment, a method of mapping a cardiac conduction system in a pediatric heart during open heart surgery using a handheld device including a rigid handle, a flexible intermediate component coupled to the handle, and a probing portion including an array of electrodes held in a fixed arrangement by a conformal electrode support coupled to the intermediate component includes placing the array of electrodes of the probing portion in contact with a first position on the heart by holding and moving the handle. The array of electrodes provides a first set of signals to processing circuitry via wires coupled, respectively, to the array of electrodes, the first set of signals indicating a level of electrical activity at the first position. The method also includes moving the array of electrodes of the probing portion to be in contact with a second position on the heart by moving the handle to raise the probing portion from the first position and holding and moving the rigid handle to place the array of electrodes at the second position based on determining that the cardiac conduction system is not located at the first position. The array of electrodes provides a second set of signals to the processing circuitry via the wires, the second set of signals indicating a level of electrical activity at the second position.

The foregoing has outlined some of the pertinent features of the disclosed subject matter. These features are merely illustrative.

### BRIEF DESCRIPTION OF THE DRAWINGS

Some examples of the disclosed technology are described making reference to the following drawings. In the figures, like-referenced numerals designate corresponding parts throughout the different views.

FIG. 1 shows a system to map an area with cardiac conduction pathways according to one or more embodiments;

FIG. 2 details aspects of a probing portion with exemplary and optional features according to one or more embodiments;

FIG. 3A shows an exemplary embodiment of the probing portion with three electrodes;

FIG. 3B shows an exemplary embodiment of the probing portion with nine electrodes;

FIG. 3C shows an exemplary embodiment of the probing portion with twelve electrodes;

FIG. 4 details aspects of the handheld device and a probe interface of the intermediate component that attaches to the probing portion according to one or more embodiments;

FIG. 5 details aspects of the handheld device and a handle interface of the intermediate component that attaches to the handle according to one or more embodiments;

FIG. 6 shows aspects of a handheld device according to one or more embodiments;

FIG. 7 is a cross-sectional view of an exemplary intermediate component according to one or more embodiments;

FIG. 8 is a cross-sectional view of wires directed along an intermediate component and through a handle according to an exemplary embodiment;

FIG. 9 is a cross-sectional view of an exemplary intermediate component according to one or more embodiments;

FIG. 10 is a cross-sectional view of wires directed through an intermediate component and through a handle according to an exemplary embodiment;

FIG. 11A illustrates a handheld device positioned within a heart according to one or more embodiments;

FIG. 11B illustrates the handheld device with the probing portion conformally contacting the heart;

FIG. 12A shows a probing portion according to an exemplary embodiment;

FIG. 12B illustrates conformability of the probing portion of FIG. 12A according to an exemplary embodiment; and

FIG. 13 is a process flow of a method of performing intraoperative endocardial conduction mapping according to one or more embodiments.

### DETAILED DESCRIPTION

Reference will now be made to the drawings to describe the present disclosure in detail. It will be understood that the drawings and exemplified embodiments are not limited to the details thereof. Modifications may be made without departing from the spirit and scope of the disclosed subject matter.

Open heart surgery may be performed to address a number of issues. Even if the surgery successfully corrects the initial problem, the procedure itself may result in life-long complications if conduction tissue in the heart is damaged by inadvertent contact during the surgical procedure. The cardiac conduction system is not visible and, thus, cannot be avoided without prior localization. Further, prediction of the location of conductive tissues is increasingly difficult in patients with complex forms of congenital heart disease. The dangers of contacting conduction tissue, as well as the impact, may be exacerbated in children. Mapping the proximal conduction tissue, including the His bundle, may facilitate avoiding mechanical damage to the area during open heart surgery. In addition, the localized conduction pathways may be used to develop and/or further train predictive models of conduction location in patients with complex congenital heart defects. For example, by performing a classification and regression tree (CART) analysis, specific anatomic factors (e.g., ventricular looping, visceroatrial situs) may be used to predict the location of the conduction system.

One approach to localizing the His bundle involves using a catheter-based cardiac electrical mapping system to localize cardiac conduction pathways. This approach involves an array of electrodes arranged on a semi-rigid support at the distal end of a catheter. The surgeon must control the extensive length (e.g., 110 centimeters) of the catheter and stabilize the electrodes against tissue. The electrical signals obtained from the electrodes are used to determine whether the array is on conduction tissue. If it is not, the surgeon must maneuver the electrodes to a different position to repeat the check. The size and shape of the array of electrodes may make sufficient contact between the electrodes and underlying tissue challenging. This is because, if the region of interest inside the heart does not closely match the shape of the array or contact is not forced by holding down the electrodes, signal quality from the tissue will be poor. In addition, the spatiotemporal resolution of the data obtained by the catheter-based device is limited.

The inventors recognized and appreciated the need for a more customized multielectrode array and more precise intraoperative endocardial conduction mapping. Rather than a long catheter-based instrument, a handheld device may be assembled with an array of electrodes at one end, according to one or more embodiments. A flexible intermediate component may connect a rigid handle to the electrode array, allowing downward pressure to be applied on heart tissue that is partly deflected by the flexible component. The pressure may ensure sufficient contact between the electrodes and heart tissue while the deflection may ensure that damage is not caused during the probing. The device may be customizable for different size and shape needs based on a number of different arrangements and sizes of electrode arrays that may be selectably put on the end of the handheld device. The electrodes of a given set may be held in a fixed arrangement by a conformal support according to one or more embodiments, allowing sufficient contact between each electrode of the array and the underlying tissue.

Aspects of the exemplary embodiments result in intraoperative mapping that accurately localizes endocardial conduction. As detailed, the size, shape, and density of an electrode array used for the mapping may be matched to a particular patient and application. In addition, the maneuverability of the device, limited deflection of a handle portion, and conformability of the support for the electrodes ensures sufficient contact between the electrodes and underlying tissue. Signals from the electrodes may be used to identify the presence of His bundle potential, which corresponds to conduction exiting the atrioventricular node and entering the proximal conduction system. As detailed, identifying and marking the area can help a surgeon avoid causing mechanical damage that leads to iatrogenic heart block or other complications. Placement of patches or sutures, resection of tissue, or other cardiac repair techniques may be planned in consideration of the location of the cardiac conduction pathways to optimize the geometric outcome of the repair while minimizing a risk of injury to the conduction system.

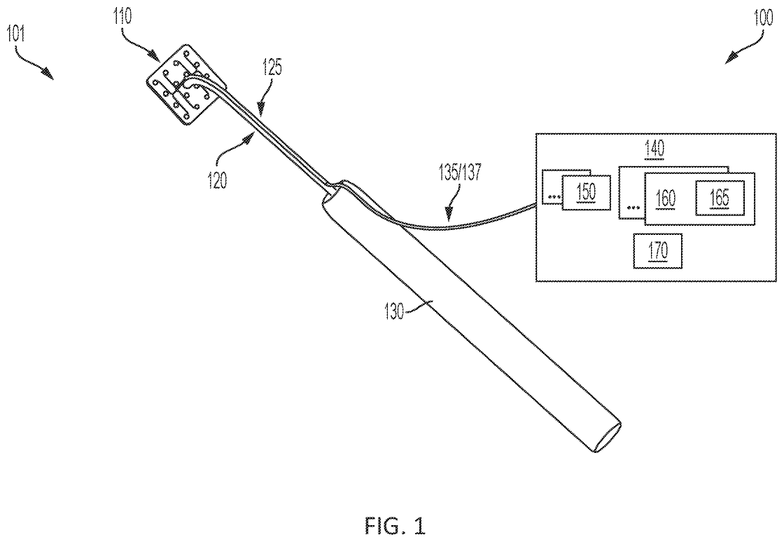

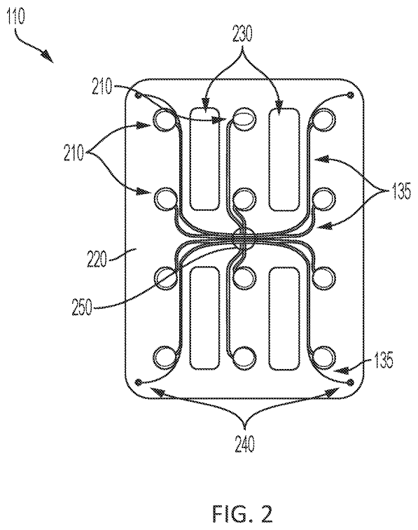

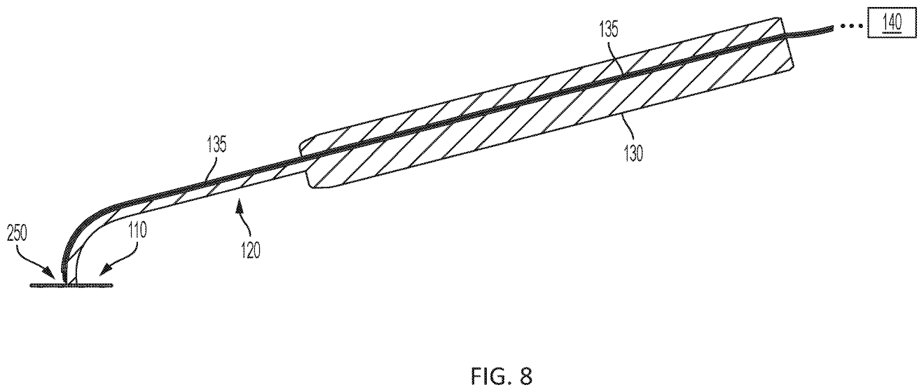

FIG. 1 shows an exemplary system 100 to map cardiac conduction pathways according to one or more embodiments. The system 100 includes a handheld device 101 and processing circuitry 140. The handheld device 101 includes an interchangeable probing portion 110 that is further discussed with reference to FIG. 2. A flexible intermediate component 120 connects the probing portion 110 to a rigid handle 130. While the overall length of the handheld device 101, from the probing portion 110 to the end of the handle 130 is not limited, it is likely to be shorter than a catheter-based probing device and may be sized for maneuverability and control (e.g., on the order of 12 centimeters (cm) to 20 cm or, generally less than 30 cm). According to an exemplary embodiment, a wire channel 125 extends the length of the intermediate component 120 from the probing portion 110 and carries one or more wires 135 between the probing portion 110 and processing circuitry 140. The wire channel 125 may be an insulated tube, for example. The wires 135 guided through the wire channel 125 may be kept in the insulated channel material in the form of a channel 137 to the processing circuitry. According to additional exemplary embodiments discussed with reference to FIGS. 8-10, the wires 135 may pass through the intermediate component 120 and/or the handle 130.

The processing circuitry 140 includes one or more processors 150 and memory 160 to process signals received from the probing portion 110, as detailed with reference to FIG. 1. Memory 160 includes a non-transitory computer-readable medium 165 that may store instructions that may be processed by one or more of the processors 150. The instructions stored by the non-transitory computer-readable medium 165 may be processed to display the signals from the probing portion 110 and, alternately or additionally, to implement one or more algorithms using the signals. The processing circuitry 140 may also include an interface 170 to facilitate display of the signals or to output information obtained from the signals in a textual or visual format.

According to exemplary embodiments, the signals carried by the wires 135 may be displayed via the interface 170 (e.g., following amplification and filtering). The signals may be analyzed (e.g., by an electrophysiologist or other member of the healthcare team) to determine if the location of the probing portion 110 (at which the signals were obtained) indicates the presence of cardiac conduction pathways. The signals and identification process may be similar to those undertaken with the catheter-based approach. Alternately or additionally, the processing circuitry may analyze the signals to determine whether the cardiac conduction pathways have been located and output the result of that determination.

FIG. 2 details aspects of a probing portion 110 with exemplary and optional features according to one or more embodiments. The exemplary probing portion 110 has twelve electrodes 210 that are held in a fixed arrangement by an electrode support 220. Generally, the electrodes 210 may be spaced at a distance on the order of 2 millimeters or more from each other. The electrode support 220 may encapsulate the electrodes 210, for example. The material and thickness of the electrode support 220 may be selected based on a degree of conformability needed for the array of electrodes 210. For example, an array of electrodes 210 that spans a larger area may require an electrode support 220 with more conformability. This is because the electrodes 210 that span the larger area may need to contact cardiac tissue at different levels or heights.

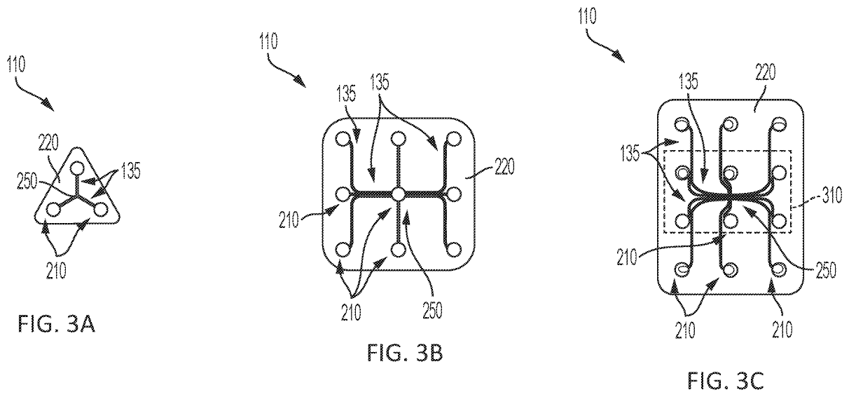

On the other hand, an array of three electrodes 210 that are closely spaced may require an electrode support 220 that is relatively more rigid to ensure close contact with underlying tissue. Additional rigidity may be achieved for the probing portion 110 in a number of ways. The wires 135 embedded in the electrode support 220 may be more rigid. Additionally or alternately, the material of the electrode support 220 may be more rigid. Exemplary and non-limiting materials for the electrode support 220 may include silicone or hydrogel, which are flexible and conformal, or mylar or polyimide, which are flexible and film-like. Further, the spacing of the electrodes 210 may, itself, affect rigidity. That is, more closely spaced electrodes 210 may result in a more rigid probing portion 110. Exemplary and non-limiting examples of electrodes 210 may include platinum-iridium, silver, or stainless steel. The surface area of each electrode 210 may be on the order of 1-1.5 square-millimeters, for example, with signal fidelity sufficient for use in a human heart. The exemplary number and arrangement of electrodes 210 in FIG. 2 is not intended to limit alternate numbers and arrangements of electrodes 210, some of which are shown in FIGS. 3A, 3B, and 3C, for example. Any of the exemplary arrangements of electrodes 210 ensures that a bipole pair is able to detect the signal wavefront.

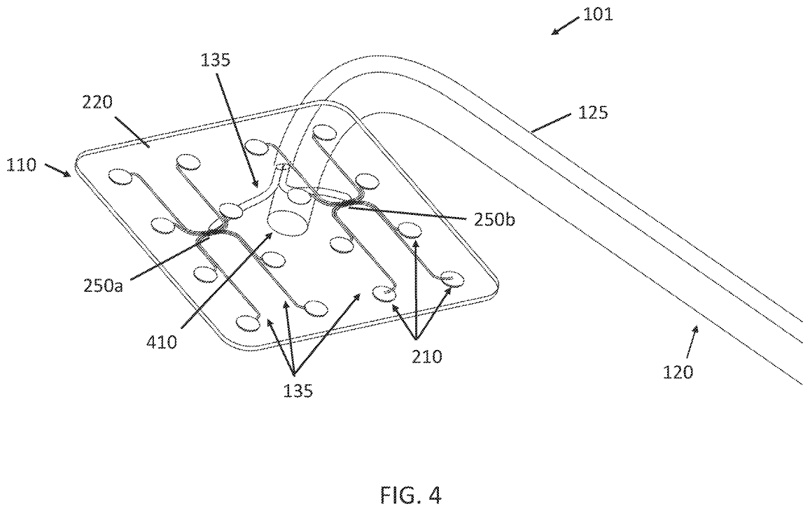

As shown in FIG. 2, a wire 135 is connected to each electrode 210. The wire 135 carries the signal indicating the electrical activity detected by the electrode 210. As shown, the wires 135 from each of the electrodes 210 are routed to a combining area 250 where they may be combined and guided through the wire channel 125 to the processing circuitry 140. As discussed with reference to FIG. 4, different sets of wires 135 may alternately be routed to different combining areas 250 to be combined. The wires 135 that are part of the probing portion 110 may be encapsulated by the electrode support 220 like the electrodes 210. The electrode support 220 may optionally include marking cutouts 230. Marking cutouts 230 refer to areas where the electrode support 220 is cut out to expose cardiac tissue under the probing portion 110 and to facilitate marking the cardiac conduction pathways with a surgical marking pen based on identification via the system 100.

Electromagnetic sensors 240 may optionally be included in different areas of the electrode support 220. Wires 135 connect the electromagnetic sensors 240 to the processing circuitry 140 in a similar manner to the wires 135 that connect the electrodes 210 to the processing circuitry 140. The wires 135 from the electromagnetic sensors 240 are also routed to the combining area 250, as shown. The potential of the electromagnetic sensors 240 may be used to track the array of electrodes 210 in three-dimensional space. That is, rather than collecting electrical potentials, like the electrodes 210, the electromagnetic sensors 240 have a potential that, when detected, denotes their location in space. Thus, through the arrangement of the electromagnetic sensors 240 around the electrode support 220, detection of their potentials facilitates visualization of a position and orientation of the probing portion 110 on the heart.

FIGS. 3A, 3B, and 3C illustrate exemplary embodiments of the probing portion 110. FIG. 3A shows an exemplary embodiment of the probing portion with three electrodes 210. The electrode support 220 is triangular in shape and the electrodes 210 are positioned at the three corners. FIG. 3B shows an exemplary embodiment of the probing portion 110 with nine electrodes 210. The electrodes 210 are positioned around the perimeter of the electrode support 220 and at its center, where the wires 135 are routed to a combining area 250. FIG. 3C shows an exemplary embodiment of the probing portion 110 with twelve electrodes 210. According to the orientation shown in FIG. 3C, the electrodes 210 are arranged in four rows of three. Increasing the number of electrodes may facilitate more bipole pairing options and increased coverage area to enhance the characterization and localization of the conduction system. The overall shape of the probing portion 110 according to various embodiments may determine suitability to different heart geometries and regions of interest. Suitability may refer, for example, to improved signal quality. Different areas of the heart may require different sizes and shapes of probing portions 110. For example, a shape that suits a ventricular septal defect (VSD) crest may be less suited for use underneath a heart valve.

According to exemplary embodiments, the arrangement of the wires 135 and material of the electrode support 220 may facilitate dynamic modification (e.g., resizing and/or reshaping) of the probing portion 110. That is, one or more outer electrodes 210 may be cut, along with at least some of the wires 135 extending from those electrodes 210, to result in a dynamically modified probing portion 310, as indicated in FIG. 3C. As shown, the six outer electrodes 210 and some portion of the wires 135 from the six electrodes 210 may be cut away to result in the modified probing portion 310.

Along with size and shape, related exemplary factors that may be used to select a probing portion 110 are time and resolution. That is, a small, closely spaced set of electrodes 210 may localize the cardiac conduction pathways with greater resolution. However, a larger array with more electrodes 210 may facilitate faster endocardial conduction mapping. Completing the mapping as quickly as possible ensures that the time of the overall surgical procedure is not significantly extended, which may generally be safer for the patient. According to exemplary embodiments, a larger probing portion 110 with more electrodes 210 may be desirable initially, while a smaller probing portion 110 providing greater resolution may be used once a region of conduction activity is identified. While the maximum number of electrodes 210 is not limited, 30 to 100 electrodes 210 may generally be an upper limit.

FIG. 4 details aspects of the handheld device 100 and a probe interface 410 of the intermediate component 120 that attaches to the probing portion 110 according to one or more embodiments. FIG. 4 shows an exemplary probing portion 110 that includes an array of sixteen electrodes 210. According to the orientation depicted in FIG. 4, the wires 135 from the eight electrodes 210 on the left are combined at combining area 250a and the wires 135 from the eight electrodes 210 on the right are combined at combining area 250b (generally referred to as combining area 250). The two combinations of wires 135 are guided via the wire channel 125 to the processing circuitry 140. In alternate embodiments, any number of different subsets of the wires 135 may be combined at any number of combining areas 250. Alternately, individual wires 135 may be routed to the wire channel 125. The flexibility in the wiring arrangement facilitates flexibility in the configuration of the electrodes 210 of the array and also facilitates dynamic modification of the probing portion 110.

FIG. 4 also shows a probe interface 410 of the intermediate component 120 that is attached to the probing portion 110. More particularly, as shown, the probe interface 410 of the intermediate component 120 may be affixed to the electrode support 220 of the probing portion 110. By disconnecting the probing portion 110 at the combining areas 250 and at the probe interface 410, a different probing portion 110 may be selected and affixed to the intermediate component 120. According to exemplary embodiments, a real-time three-dimensional model of the heart undergoing an operation may be generated. A size and shape match may be performed between the three-dimensional model of the heart and different configurations of electrodes 210 of different probing portions 110 to select the best-suited probing portion 110 for a specific operation.

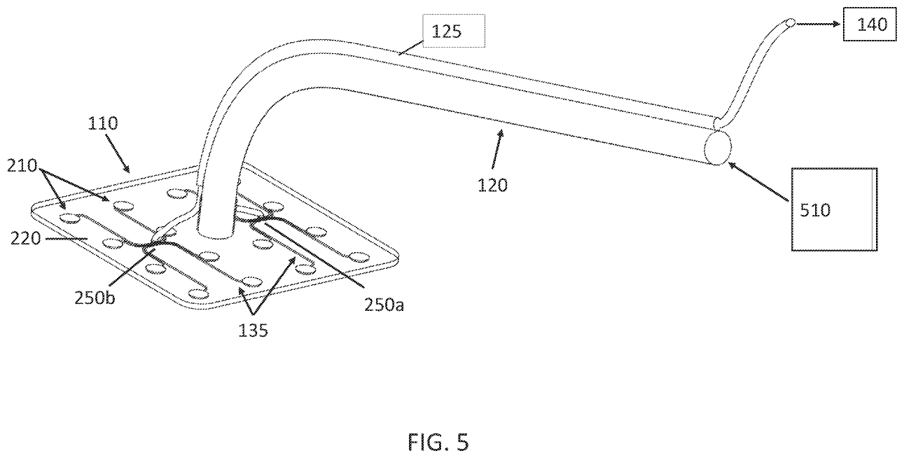

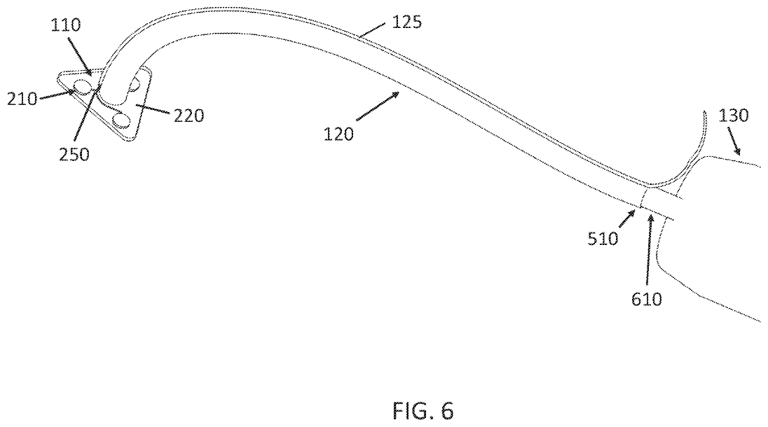

Alternately, the size and shape matching may involve different configurations of electrodes 210 of different (complete) handheld devices 101. As discussed with reference to FIGS. 5 and 6, according to yet another alternative embodiment, size and shape matching may involve selecting a particular probing portion 110 and flexible component 120 combination to attach to a handle 130. In addition to size and shape, density of electrodes 210 may be another consideration in the selection of a probing portion 110, regardless of whether the probing portion 110 is selected alone, in combination with the intermediate component 120, or as a full handheld device 101. Multiple electrode densities may be required to optimize signal integrity and to maintain a minimum number of electrodes 210 needed to ensure distinguishability of the His bundle potential in varying cardiac geometries. As previously noted with reference to FIG. 3C, size and shape matching may involve dynamically modifying (e.g., cutting) the probing portion 110 to obtain a modified probing portion 310.

FIG. 5 details aspects of the handheld device 100 and a handle interface 510 of the intermediate component 120 that attaches to the handle 130 according to one or more embodiments. The probing portion 110 shown in FIG. 5 is the same one shown in FIG. 4. Thus, as discussed with reference to FIG. 4, there are two combining areas 250a, 250b for the wires 135 from the various electrodes 210 used to channel all the wires 135 into the wires channel 125. FIG. 5 shows the intermediate component 120 detached from the handle 130 to expose the handle interface 510, which is the end of the intermediate component 120 that is opposite the end of the intermediate component 120 that include the probe interface 410 shown in FIG. 4.

FIG. 6 shows aspects of a handheld device 100 according to one or more embodiments. The probing portion 110 shown in FIG. 6 differs from that in FIG. 5. Because the probing portion 110 shown in FIG. 6 only includes three electrodes 210, only three wires 135 are guided through the wire channel 125 of the intermediate component 120. In FIG. 6, the handle interface 510 of the intermediate component 120 is shown attached to the handle 130. The attachment may be accomplished in one of a number of ways. According to an exemplary embodiment, the handle interface 510 is hollow and fits onto a protrusion 610 on the handle 130. According to another exemplary embodiment, the protrusion 610 on the handle 130 is hollow, and the handle interface 510 of the intermediate component 120 fits into the protrusion 610. In either case, the intermediate component 120 is easily detached from the handle 130. As such, different probing portion 110 and intermediate component 120 combinations (e.g., the probing portion 110 and intermediate component 120 shown in FIGS. 4 and 5) may be selectably used with the same handle 130. According to alternate embodiments, the handle interface 510 of the intermediate portion 120 is affixed to the protrusion 610 of the handle 130 or to a handle 130 without a protrusion 610.

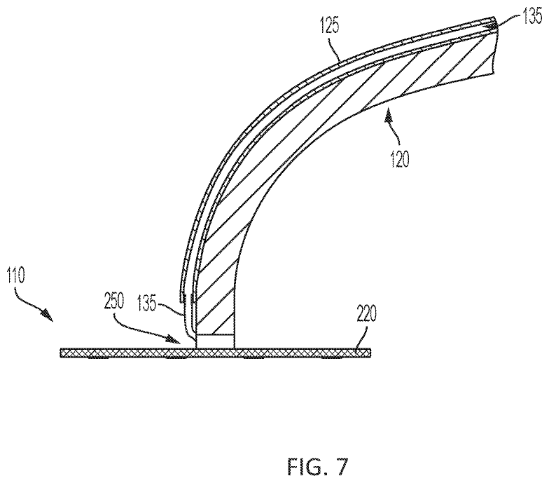

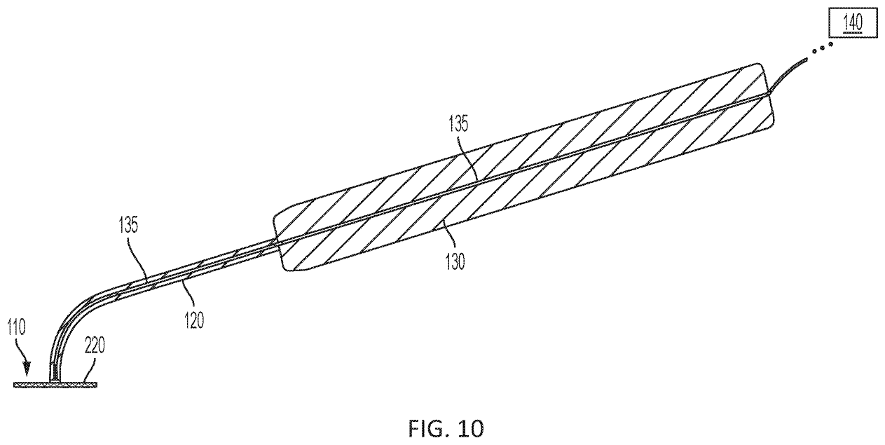

FIG. 7 is a cross-sectional view of an exemplary intermediate component 120 according to one or more embodiments. The cross-sectional view in FIG. 7 details wires 135 in a wire channel 125 of the intermediate component 120. Wires 135 from a combining area 250 of the probing portion 110 extend through the wire channel 125, as shown. According to an exemplary embodiment, the wires 135 are directed from the intermediate component 120 to the processing circuitry 140 (e.g., as shown in FIG. 1). According to alternate embodiments shown in FIGS. 8 and 10, the wires 135 may extend through the length of the handle 130, as well.

FIG. 8 is a cross-sectional view of wires 135 directed along an intermediate component 120 and through a handle 130 according to an exemplary embodiment. The wires 135 may be directed through a wire channel 125, as shown in FIGS. 1 and 7. The wires 135 may be directed along the intermediate component 120 from a combining area 250 of the probing portion 110. The wires 135 emerging through the handle 130 may be directed to the processing circuitry 140, as indicated.

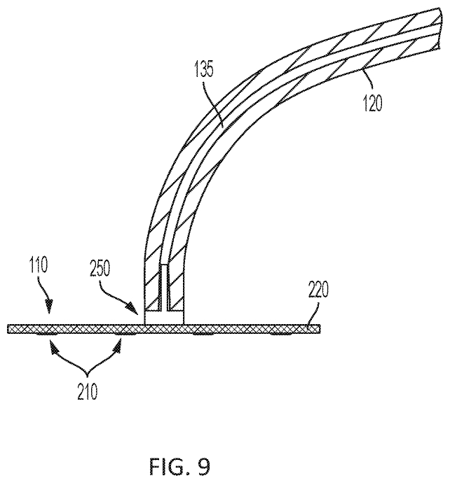

FIG. 9 is a cross-sectional view of an exemplary intermediate component 120 according to one or more embodiments. The cross-sectional view of FIG. 9 details wires 135 extending through an intermediate component 120. Wires 135 from a combining area 250 of the probing portion 110 extend through the intermediate component 120 rather than in a wire channel 125, as shown in FIGS. 1 and 7, for example.

FIG. 10 is a cross-sectional view of wires 135 directed through an intermediate component 120 and through a handle 130 according to an exemplary embodiment. The wires 135 may be directed from a combining area 250 of the probing portion 110 into the intermediate component 120. The wires emerging from the handle 130 may be directed to the processing circuitry 140, as indicated. Although not specifically shown, the wires may be directed through a channel along the handle 130 in a similar manner to being directed through the wire channel 125 of the intermediate component 120.

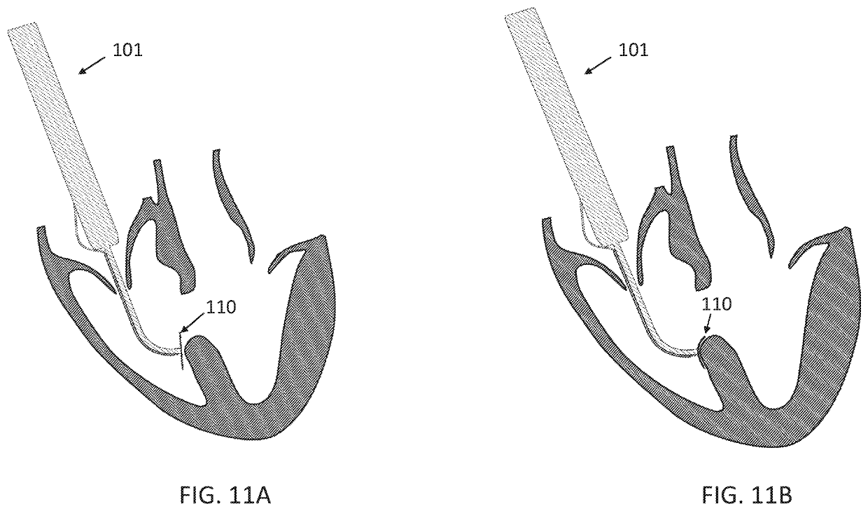

FIGS. 11A and 11B show a handheld device 101 during use in a heart with a ventricular septal defect (VSD) according to one or more exemplary embodiments. FIG. 11A shows the handheld device 101 with the probing portion 110 inserted through a tricuspid valve orifice of the heart in preparation for conduction mapping. FIG. 11B shows the handheld device 101 with the probing portion 110 conformally contacting the VSD crest. As FIG. 11A shows, the handheld device 101 may be held such that no portion of the handheld device 101 contacts any portion of the patient. Even during probing, only the probing portion 110 may be in contact with the heart or any portion of the patient, which is not the case in a catheter-based device. Generally, to probe different areas of the heart to locate the conduction system, the probing portion 110 may remain in contact with the heart and be moved along the endocardial surface of the heart, but the probing portion 110 can be taken completely out of contact with the heart via control of the handle 130.

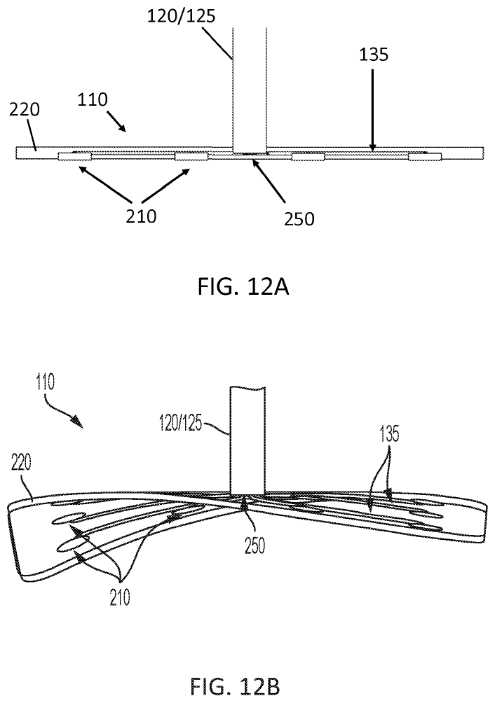

FIG. 12A shows an exemplary probing portion 110 that may be used in a handheld device 101 according to exemplary embodiments described herein. The exemplary probing portion 110 includes twelve electrodes, like the embodiment shown in FIG. 3C. According to the orientation shown in FIG. 12A, the intermediate component 120 is not visible (e.g. is behind the wire channel 125). As shown, the electrode support 220 is flat (i.e., each of the electrodes 210 is at the same level). FIG. 12B illustrates conformability of the probing portion 110 of FIG. 12A according to an exemplary embodiment. As FIG. 12B illustrates, each of the electrodes 210 may be at a different level, relative to the base level shown in FIG. 12A, based on the flexibility of the electrode support 220. This conformability of the array of electrodes 210 facilitated by the conformability of the electrode support 220 facilitates a sufficient contact between each electrode 210 and underlying tissue. The material composition and thickness of the electrode support 220 may be controlled to control a degree of conformability of the array of electrodes 210.

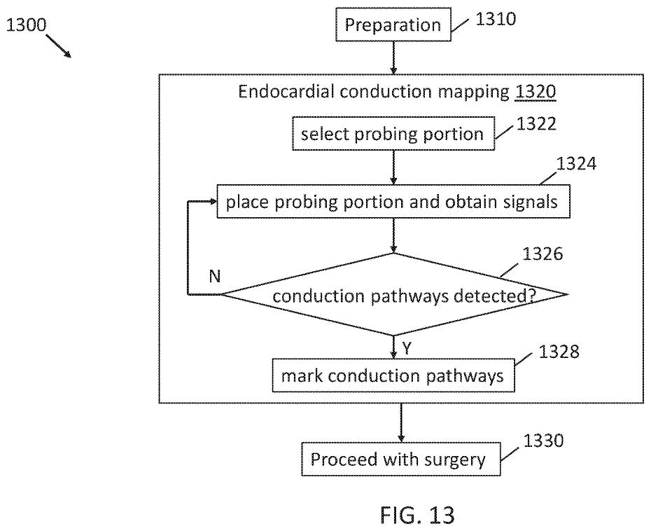

FIG. 13 is a process flow of a method of performing intraoperative endocardial conduction mapping according to one or more embodiments. At 1310, preparation may entail arterial cannulation to provide oxygenated blood to bypass the heart, as well as inducing ventricular fibrillation to eliminate cardiac ejection (i.e., the heart pumping out blood). The heart may then be opened via an atriotomy (i.e., opening of an atrium) or ventriculotomy (incision into one or both ventricles). Once cardiotomy suckers are placed across the atrioventricular valves to ensure that the heart cannot pressurize or eject blood, a defibrillator may be applied to put the heart back in normal sinus rhythm, which refers to the resumption of cardiac conduction.

At 1320, endocardial conduction mapping refers to processes involved in using the handheld device 101 and the system 100, generally, to localize cardiac conduction pathways. At 1322, selecting a probing portion 110 may refer to selecting from among a set of handheld devices 101, selecting from among a set of combinations of the probing portion 110 and intermediate component 120 that will be affixed to a handle 130, or selecting from among a set of probing portions 110 that will be affixed to an intermediate component 120 and handle 130. The selection may be based on a size or age of the patient. According to exemplary embodiments, the selection may be based on matching a size and shape of available probing portions 110 to a desired size and shape that are determined from a three-dimensional model of the patient's heart.

At 1324, placing the probing portion 110 (selected at 1322) and obtaining signals refers to signals from the electrodes 210 of the probing portion 110 being transferred over wires 135 to the processing circuitry 140 while the electrodes 210 are at a particular position on the heart. The placement at the particular position is achieved by a surgeon holding the handle 130 of the handheld device 101 and positioning the probing portion 110 at the particular position such that the electrode support 220 is conformally draped over the particular position of the heart and each of the electrodes 210 is in contact with the heart at the particular position. At 1326, the processes include checking whether conduction pathways are detected at the location where the probing portion 110 was placed when the signals were obtained (at 1322). For example, the signals provided by the electrodes 210, which indicate a level of electrical activity, may be viewed via the interface 170 to determine if any of the signals meet the criteria to indicate a His bundle potential (e.g. based upon signal amplitude and temporal position within the electrogram).

If the check at 1326 indicates that conduction pathways are not detected, the processes of placing the probing portion 110 at a different position and obtaining signals (at 1324) may be repeated iteratively. If the check at 1326 indicates that conduction pathways are detected, then marking conduction pathways, at 1328, may involve using marking cutouts 230 in the electrode support 220 to mark the underlying cardiac tissue with a surgical pen. Once the conduction pathways are marked or otherwise identified (at 1328), proceeding with the surgery, at 1330, refers to completing the corrective procedure on the heart while avoiding the conduction pathways that were mapped according to the endocardial conduction mapping processes (at 1320). Once it is determined (by the check at 1326) that the cardiac conduction pathways have been located and marked as desired, the handheld device 100 may be easily separated/removed from the heart to continue the procedure (at 1330).

Although explanatory embodiments have been described, other embodiments are possible. Variations on the exemplary methods, including re-ordering and omission or modification of some processes, are contemplated, and such variations are within the scope and spirit of the embodiments detailed herein.

What is claimed is:

1. A system to map a cardiac conduction system during open heart surgery, the system comprising:
an electrode support;
electrodes held in a fixed arrangement by the electrode support;
a flexible intermediate component, wherein the electrodes and the electrode support are affixed to a first end of the intermediate component; and
a rigid handle attached to a second end of the intermediate component, opposite the first end.

2. The system according to claim 1, wherein a length of a combination of the handle and the intermediate component is less than or equal to 30 centimeters.

3. The system according to claim 1, wherein a number of the electrodes ranges from three to one hundred.

4. The system according to claim 1, further comprising wires, wherein each of the electrodes is coupled to one of the wires and the electrodes and the wires are encapsulated by the electrode support.

5. The system according to claim 4, wherein the electrode support material is conformal.

6. The system according to claim 1, further comprising wires, wherein each of the electrodes is coupled to one of the wires and the wires are connected to processing circuitry.

7. The system according to claim 6, wherein the wires are directed along the intermediate component within a channel of the intermediate component or through the intermediate component.

8. The system according to claim 6, wherein the wires are directed through the handle.

9. The system according to claim 1, wherein adjacent ones of the electrodes are 2 millimeters or more from each other.

10. A handheld cardiac conduction mapping system, comprising:
a rigid handle;
a flexible intermediate component coupled to the handle; and
a set of probing portions including at least one probing portion, each probing portion of the set of probing portions comprising:
an array of electrodes, and
an electrode support, wherein the array of electrodes is encapsulated in the electrode support.

11. The handheld cardiac conduction mapping system according to claim 10, wherein each probing portion of the set of probing portions further comprises wires, and each of the electrodes of the array of electrodes is coupled to one of the wires.

12. The handheld cardiac conduction mapping system according to claim 11, wherein the intermediate component includes a channel to direct the wires from the array of electrodes to processing circuitry.

13. The handheld cardiac conducting mapping system according to claim 11, wherein the wires are directed through the intermediate component and the handle to the processing circuitry.

14. The handheld cardiac conduction mapping system according to claim 11, wherein the electrode support is conformal.

15. A method of mapping a cardiac conduction system in a pediatric heart during open heart surgery using a handheld device including a rigid handle, a flexible intermediate component coupled to the handle, and a probing portion including an array of electrodes held in a fixed arrangement by a conformal electrode support coupled to the intermediate component, the method comprising:
placing the array of electrodes of the probing portion in contact with a first position on the heart by holding and moving the handle, the array of electrodes providing a first set of signals to processing circuitry via wires coupled, respectively, to the array of electrodes, the first set of signals indicating a level of electrical activity at the first position; and
moving the array of electrodes of the probing portion to be in contact with a second position on the heart by moving the handle to raise the probing portion from the first position and holding and moving the rigid handle to place the array of electrodes at the second position based on determining that the cardiac conduction system is not located at the first position, wherein the array of electrodes provides a second set of signals to the processing circuitry via the wires, the second set of signals indicating a level of electrical activity at the second position.

16. The method according to claim 15, further comprising selecting the probing portion from among a set of probing portions based on determining that the probing portion matches a size and geometry of a three-dimensional model of the heart more closely than other probing portions among the set of probing portions.

17. The method according to claim 15, wherein determining that the cardiac conduction system is not located at the first position is based on the first set of signals.

18. The method according to claim 15, further comprising moving the rigid handle to raise the probing portion out of contact with the first position and continuing the open heart surgery based on determining that the cardiac conduction system is located at the first position

19. The method according to claim 15, further comprising sequentially moving the array of electrodes of the probing portion to be in contact with additional positions on the heart and to generate additional sets of signals from the array of electrodes until a determination is made that the cardiac conduction system is identified.

20. The method according to claim 19, wherein determining that the cardiac conduction system is identified includes comparing the first set of signals, the second set of signals, and the additional sets of signals.
